(19) United States
(12) Patent Application Publication
(10) Pub. No.: US 2010/0297239 A1
(43) Pub. Date: Nov. 25, 2010

(54) OSSEOINTEGRATIVE MENISCUS AND CARTILAGE IMPLANTS BASED ON BETA-GLUCAN NANOCOMPOSITES

(76) Inventor: Paul GATENHOLM, Blacksburg, VA (US)

Correspondence Address: LATIMER INTELLECTUAL PROPERTY LAW, LLP, P.O. BOX 711200, HERNDON, VA, 20171, US

(21) Appl. No.: 12/645,453

(22) Filed: Dec. 22, 2009

Related U.S. Application Data
(60) Provisional application No. 61/140,014, filed on Dec. 22, 2008.

(51) Int. Cl. A61K 9/14; C12P 19/04; A61P 19/04

(52) U.S. Cl. 424/488; 435/101

(57) ABSTRACT

The present invention provides engineered composite materials for use in medical treatment of injured or degenerated menisci, cartilage, and bone. The composite materials include a cellulosic layer substantially or completely consisting of β-1→4-glucan units, and a hydrogel layer substantially or completely consisting of copolymers of β-1→2-glucan, β-1→3-glucan, and/or β-1→4-glucan, or mixtures of two or all three of these units. Production of the composite materials is achieved in a single culture milieu, using regulation of oxygen availability to control production of the various units and deposition of the layers by Acetobacter xylinum or other microorganisms that produce extracellular cellulosic material.

### CROSS-REFERENCE TO RELATED APPLICATIONS

The present application relies on and claims the benefit of the filing date of U.S. provisional patent application No. 61/140,014, filed 22 Dec. 2008, the entire disclosure of which is hereby incorporated herein by reference.

### BACKGROUND OF THE INVENTION

1. Field of the Invention

The present invention relates to the fields of medicine and microbiology. More specifically, the present invention relates to microbially-produced composite materials for use as medical implants to treat meniscus and cartilage damage.

2. Description of Related Art

Over fifteen million people worldwide suffer from knee joint failure each year due to the breakdown of surrounding cartilage in the joint and the inability of this cartilage to repair itself through the natural regenerative processes of healing in the body. About 200,000 total knee replacements are performed each year in the U.S. at an average cost of $25,000 each, in an attempt to treat this problem.

Traumatic or degenerative meniscal lesions are a frequent problem. Meniscus cannot regenerate after resection. Therefore, meniscal lesions often progress and lead to osteoarthritis, particularly when left untreated. According to industry estimates, more than 225,000 people worldwide underwent arthroscopic meniscal repair in 2002. The menisci of the knee, which are C-shaped fibrocartilaginous tissue, perform an integral role in the motion and the preservation of knee joint by providing load bearing, shock absorption, joint lubrication, and the total stabilization of the knee joint. Meniscus injuries can be due to sport activities, car accidents, or due to general degeneration associated with aging. In the past, the lesions were managed by total resection of the injured meniscus. Nowadays the lesions are partially resected arthroscopically. The inner part of the meniscus is avascular (no blood vessels) and has therefore no ability to heal or regenerate. Clinical practices available today do not adequately regenerate meniscus. Total and partial meniscectomy (removal of meniscus) elicits degenerative changes in the affected joint or compartment.

Cellulose (β-1→4-glucan) is the most abundant polymer of natural origin. In addition to being synthesized in vast amounts as a structural material in the walls of plants, cellulose can also be produced as an exopolysaccharide, for example as bacterial cellulose (BC) synthesized by Acetobacter xylinum. Bacterial cellulose is attractive as a biomaterial because of its good mechanical properties (Backdahl et al. 2006), hydroexpansivity (Gelin et al. 2007), biocompatibility (Helenius et al. 2006), and structural stability within a wide range of temperatures and pH levels.

BC is an emerging biomaterial and several commercial products have already been registered (e.g., Biofill®, Gengiflex®). Microbially-derived cellulose in the medical industry has already been used for liquid-loaded pads, wound dressings (Fontana, et al. 1990), and other external applications. Nonetheless, bacterial cellulose has interesting properties in its wet, unmodified state. The high water content of bacterial cellulose, around 99%, suggests that it can be used as a hydrogel, which is known for its favorable biocompatible properties and lack of protein adsorption. BC is a versatile material that can be easily manufactured in various sizes and shapes. Its physical properties make it extremely attractive as an implant for biomedical applications, such as bone graft material, vascular grafts (Klemm et al. 2001), or as a hydrophilic coating of other biomaterials.

The synthesis of cellulose in Acetobacter xylinum takes place between the outer wall and the cytoplasmic membrane. Cellulose is a product of carbon metabolism and, depending on the physiological state of the cell, involves either the pentose phosphate cycle or the Krebs cycle coupled with glucogenesis. The growing glucan chains are believed to aggregate and are exported through catalytic sites that are linearly arranged on each cell (Brown et al. 1976). Acetobacter xylinum assembles glucan chains into microfibrils and subsequently into a ribbon configuration (see FIG. 1). While it is not clear why Acetobacter xylinum generates a network of cellulose, a generally accepted hypothesis is that it helps them to become floatable on oxygen-rich surfaces.

The high water holding capacity of BC results from the presence of hydroxyl groups on the surfaces of cellulose fibrils. The presence of these hydroxyl groups is likely one of the main reasons for the good biocompatibility of this material. The presence of hydroxyl groups on the surface of cellulose nanofibrils also offers the possibility for surface modifications. The state of water has been studied using dielectric spectroscopy (Gelin et al. 2007).

BC has also been evaluated as potential scaffold for tissue engineering of cartilage. For example, the present inventor and collaborators reported that bovine chondrocytes can attach to nanofibrils of cellulose (Svensson et al. 2005) (see FIG. 2). Furthermore, the present inventor and collaborators produced meniscus implants (Bodin et al. 2007c). However, it has been determined that the mechanical properties of a meniscus were difficult to mimic using the bacterial cellulose implant (i.e., the pig meniscus shown on the left in FIG. 3). This was partly due to difficulties with controlling the direction of fibrils, which is crucial for the characteristic mechanical behavior of the meniscus. However, bacterial cellulose remains a very attractive biocompatible material that can be readily formed into the necessary shapes for tissue engineering applications.

One of the challenges of using bacterial cellulose as a biomaterial and scaffold for tissue engineering has been its relatively tight structure of network of cellulose nanofibrils. The present inventor has observed that chondrocytes will typically colonized the surface of the BC material, but will not migrating into the network at an equivalent rate. Attempts have been made to induce the migration of smooth muscle cells (SMC) into the BC network using chemical attractants. Results showed that SMC were only able to enter 10 micrometers. Recently, a new technology using porogens has been developed (Backdahl et al. 2008). This technique resulted in the preparation of microporous structures that are very promising for future applications as scaffolds for tissue engineering (see FIG. 4).

In European patent application EP 1 779 875 A1, an artificial meniscus based on a hydrogel having a semi-interpenetrating network structure based on synthetic or natural polymers is described. Such materials have, however, very limited mechanical properties. They have also been shown to be difficult to attached by sutures. As suturing is a standard means for attaching bodily tissue during surgical treatment of certain injuries, the shortcoming of these menisci is substantial. In addition, Kobayashi et al 2005 reported a two year in vivo study of polyvinyl alcohol hydrogel artificial meniscus. These authors concluded that the artificial meniscus showed clinical drawbacks.

### SUMMARY OF THE INVENTION

The present invention provides biocompatible cellulosic materials, methods of making those materials, and methods of using those materials. The materials can generally be described as nanocellulose fibril reinforced β-glucan hydrogels. The materials comprise a cellulose component and a hydrogel component, the combination providing a strong, stiff, and yet compression resistant product that has numerous uses in vivo, such as, for example, meniscus replacement, cartilage replacement, and as a scaffold for tissue replacement in general, including, but not limited to bone replacement. The materials of the invention are produced by bacteria as extracellular fibrils and matrices under controlled growth conditions. The controlled growth conditions allow for regulation of the production of the cellulose fibrils and β-glucan hydrogels in a single culture medium using one or more bacterial species for production of both. The materials of the invention show high biocompatibility and are thus well suited for use in vivo in medical procedures for meniscus repair or replacement, cartilage repair or replacement, and bone repair or replacement, among other uses.

In a first general aspect, composite materials are provided. The composite materials comprise a cellulose fibril component and a β-glucan hydrogel component. The cellulose component comprises β-1→4-glucan units, which can be present in entangled fibers, which can be, in some embodiments, partially or substantially directionally oriented. The β-glucan hydrogel component comprises copolymers of β-1→2-glucan, (β-1→3-glucan, and β-1→4-glucan units. The copolymers can comprise the various glucan units in any combination and proportions.

In another general aspect, the invention provides a method of making the composite materials of the invention. In general, the method comprises growing a microorganism that is capable of producing extracellular cellulosic materials and glucan-based hydrogel materials in a suitable growth medium that allows for extracellular production of these materials. Growth of the microorganism and production of the materials is regulated to control the type of materials produced. Regulation is achieved primarily through control of the amount of oxygen made available to the microorganism. It has been found that growth in high oxygen tensions allows for production of β-1→4-glucan units predominantly, if not completely, whereas growth in low oxygen tensions allows for production of copolymers of β-1→2-glucan, β-1→3-glucan and β-1→4-glucan predominantly, if not completely. Accordingly, by controlling the amount of oxygen available to the microorganism, composite materials having finely controlled amounts of cellulose fibril and hydrogel components can be achieved. In exemplary embodiments, Acetobacter xylinum is the microorganism used to produce the composite materials.

In yet another general aspect, methods of treating subjects are provided. In general, the methods are methods of medical treatment for tissue injury or degeneration. The methods broadly comprise implanting a composite material according to the invention into a site of injury or degeneration in a subject's body. According to this aspect of the invention, the composite material can be implanted by way of any suitable procedure. Typically, some form of surgery is used to securely implant the composite material at a desired site. Exemplary methods include, but are not limited to, meniscus repair or replacement, cartilage repair or replacement, and bone repair or replacement.

Additional aspects and embodiments of the invention are discussed in more detail below.

### BRIEF DESCRIPTION OF THE DRAWINGS

The accompanying drawings, which are incorporated in and constitute a part of this specification, illustrate several features and embodiments of the invention, and together with the written description, serve to explain certain principles of the invention.

FIG. 1 is an electron micrograph showing glucan chains produced by Acetobacter xylinum and assembled into microfibrils and subsequently into a ribbon configuration.

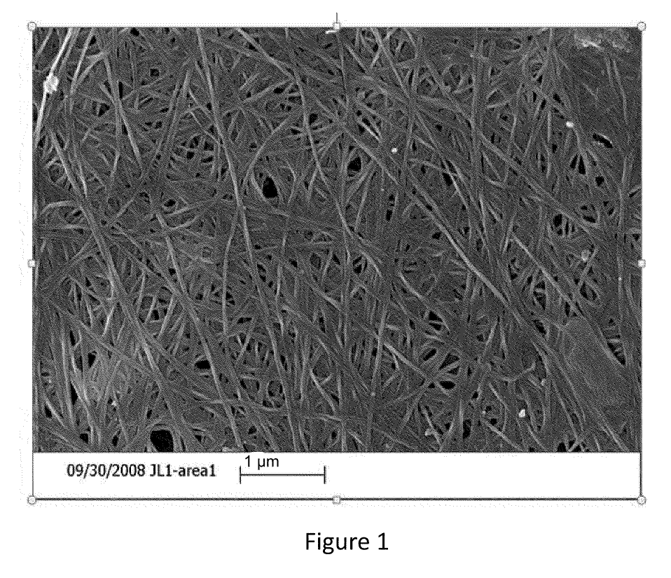

FIG. 2 is an electron micrograph of a bovine chondrocyte attached to nanofibrils of cellulose.

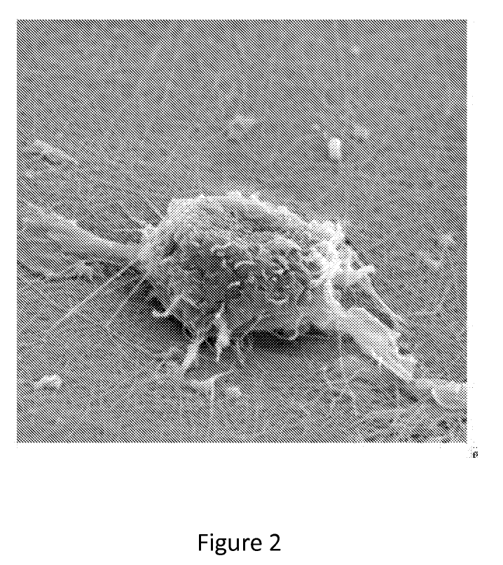

FIG. 3 is a photograph of menisci, showing a pig meniscus on the left and a bacterially-produced meniscus according to the prior art on the right.

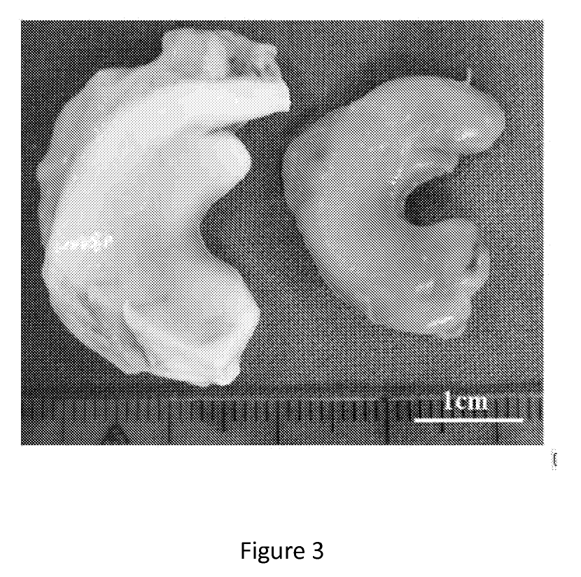

FIG. 4 is an electron micrograph of a bacterial cellulose scaffold produced using porogens.

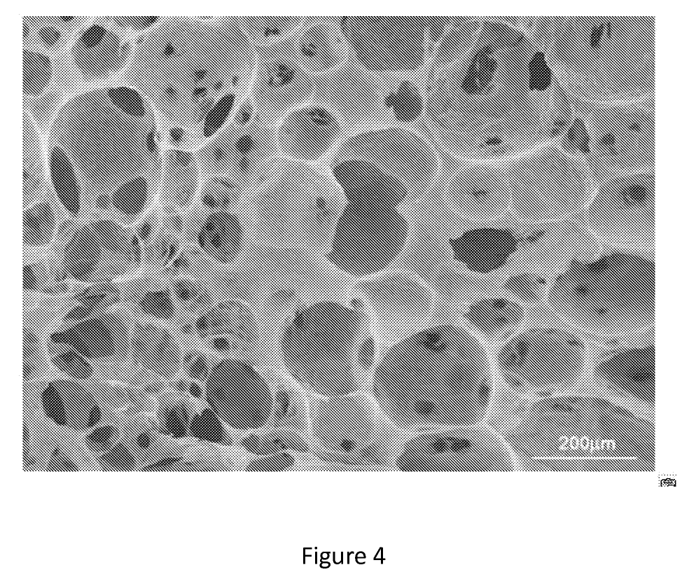

FIG. 5 shows a schematic cartoon (Panel A) and photographs (Panel B) of the morphology of cartilage, with 4 different zones depicted.

FIG. 6 shows a schematic cartoon indicating how control of bacterial fermentation can be used for preparation of cartilage replacements according to the present invention.

FIG. 7 shows the effect of carboxymethyl cellulose and calcium chloride treatment on production of crystals.

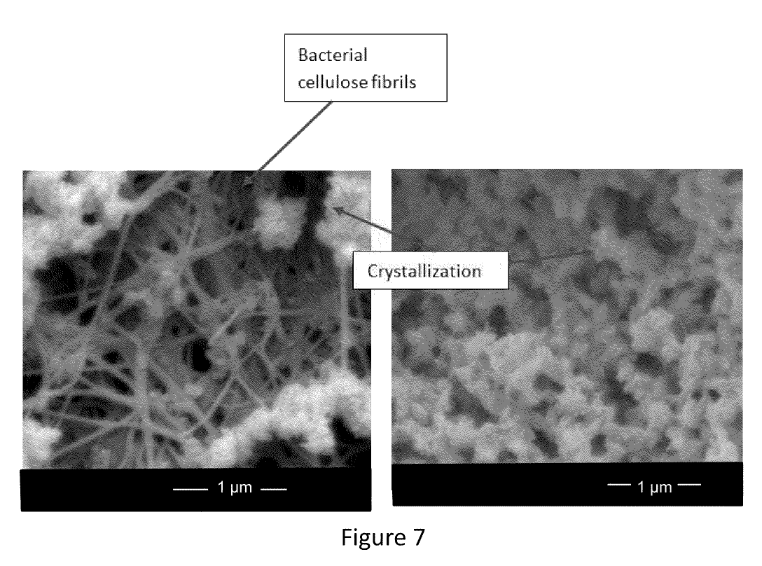

FIG. 8 shows in cartoon fashion a bioreactor for production of composite materials according to the present invention.

### DETAILED DESCRIPTION OF VARIOUS EMBODIMENTS OF THE INVENTION

Reference will now be made in detail to various exemplary embodiments of the invention, examples of which are illustrated in the accompanying drawings. It is to be understood that the following detailed description is provided to give the reader a better understanding of certain features and details of the invention, and is not intended as a limitation on the scope or content of the invention.

The present invention relates to a design and manufacturing process for β-glucan nanocomposite materials using fermentation of bacteria, such as Acetobacter, Agrobacterium, Rhizobium, Pseudomonas, and Alcaligenes, most preferably species of Acetobacter xylinum or Acetobacter pasteurianus. The most preferred strain is Acetobacter xylinum subsp.sucrofermentas BPR2001, trade number 700178™, from the ATCC. The composite materials have numerous uses, such as in applications for osseointegrative implants, particularly meniscus implant and cartilage tissue replacement. Control of production of β-1→4-glucan by bacteria is achieved by targeted oxygen delivery. It is reported herein that in high oxygen tensions, Acetobacter xylinum produces high concentrations of β-1→4-glucan (cellulose) in the form of entangled nanofibrils. These fibrils can be at least partially oriented by design of the bioreactor in or on which they are created. In contrast, under oxygen deficiency bacteria produce instead of β-1→4-glucan (cellulose nanofibrils) a clear hydrogel comprised or composed of copolymers of β-1→2-glucan, β-1→β-glucan and β-1→4-glucan. These copolymers can easily be cross-linked, if desired, to provide a material with compression resistance. The amount of cross-linking is related to the amount of compression resistance obtained. In exemplary embodiments, targeted oxygen delivery is achieved by delivering oxygen-enriched air using porous glass capillaries, although other porous materials can be used. Likewise, a syringe or other similar device may be used. Preferably, the porous material used is capable of being formed into various shapes, as desired for each particular application, or is capable of being fabricated in a pre-defined three-dimensional shape. In addition, it is preferable that the porous material be removable from the composite material when complete, for example by comprising one or more biodegradable materials that can be removed by the natural functioning of a body into which it is implanted. Alternatively, oxygen delivery can be controlled using a weak electrical filed (below about 1 V) to deliver oxygen through an electrolysis process.

The composite or nanocomposite (the terms are used interchangeably herein) described in this invention is generally a nanocellulose fibril reinforced β-glucan hydrogel. The composite is produced in a one step fermentation process, meaning that a single culture of microorganisms in a single culture vessel is used to produce the composite material. Under the production process of the invention, a nanofibril component, which provide strength and stiffness, is produced under a pre-defined oxygen tension, and a hydrogel matrix component, which provides compression resistance, is produced under a different pre-defined oxygen tension. The composite is thus achieved by targeted oxygen delivery to the microorganisms in culture. The nanocomposite prepared by this process has unique composition and it is completely produced by bacteria in one step fermentation process. The nanocomposite exhibits similar biomechanical properties as native cartilaginous tissue, which is composed of collagen nanofibrils reinforcing a proteoglycan hydrogel. The production process can be modulated with the consequent controlled variation of the composition of the nanocomposite.

As a consequence of certain bioreactor designs for targeted oxygen delivery, β-1→4-glucan nanocomposites described in this disclosure show a microporosity at the edges. This feature or characteristic can be used to stimulate the osseointegration process. Furthermore, the microporous nanocomposite described in this disclosure, when treated with acidic polysaccharides such as carboxymethylcellulose and placed in simulated body fluid has the ability to induce crystallization of hydroxyapatite, varying in size from nano to micro. Such hydroxyapatite decorated cellulose nanofibrils with well defined microporosity stimulate migration, attachment, and differentiation of osteblasts, osteprogenitor cells, chondrocytes, and stem cells, which result in bone tissue and thus osseointegration of the composite material when implanted into a subject.

While the practical applications of the composite materials of the invention are numerous, exemplary uses lie in the regenerative medicine field, for example for meniscus replacement, cartilage repair, and cartilage tissue generation. Any type of meniscus replacement can be accomplished, including but not limited to meniscus of the knee. Furthermore, any type of cartilage can be repaired or replaced, such as ear and nose tissue. Likewise, any type of bone tissue can be repaired or replaced, including but not limited to tibia, fibula, patella, femur, bones of the pelvis, bones of the spine and neck, ribs, clavicle, mandible, bones of the cranium, humerus, radius, and ulna. It is to be understood that the present invention has applicability in both humans and animals. While the disclosure focuses on human applications, the applicability to animals is evident.

This present innovation describes a new method of fermentation in which bacteria produce oriented cellulose nanofibrils with high density and at the same time β-glucan hydrogel is produced. More specifically, it has been found that at normal oxygen concentrations nanofibers are produced. The increased oxygen tension causes production of more cellulose nanofibrils. Oxygen deficiency switches production from cellulose to glucan hydrogel.

As mentioned above, the invention provides composite materials comprising a cellulose fibril component and a β-glucan hydrogel component. In general, the composites comprise the components in a laminate configuration, having one component layered on top of the other component. While the composite material can be provided in a sheet-like configuration, for ease of description, it is described herein with respect to a more complex three-dimensional configuration, having an interior and an exterior rather than simply a top and bottom. It is to be understood that the concepts described with regard to the complex three-dimensional configuration are fully applicable to the lesser complex sheet-like configuration. In an exemplary configuration, the composite comprises a layer of cellulosic material in fibril form surrounded on its exterior surface by a layer of hydrogel material. The two layers are deposited by way of the action of a microorganism, such as Acetobacter xylinum, which produces the layers extracellularly. Exemplary composite materials comprise a single layer of each component, similar to naturally produced cartilagenous materials. However, the composites of the invention are not limited to two-layer structures. Rather, more complex layered structures are envisioned by the invention. As such, a composite material having three, four, five, six, or more layers can be produced according to the present invention.

The composite materials of the invention are not limited in the particular amounts of each component. That is, there is no limitation on the relative thicknesses of the various components of the composite materials, and the relative amounts can be varied depending on the desired use for the particular material produced. In exemplary embodiments, when viewed in cross-section, the cellulosic component has a lesser height than the hydrogel component. In embodiments, the cellulosic component is less than one-half of the height of the hydrogel component, such as one-third of the height or one-fourth of the height. In other embodiments, the cellulosic component represents less than 50% of the dry volume of the composite material. For example, it can represent 40% or less of the total volume, 30% or less of the total volume, 20% or less, or 10% or less. Typical bacterial cellulose material is 99% water and 1% cellulose. In this case the cellulose content is lower and replaced by glucan hydrogel.

Composite materials according to the invention can take any shape or size. Preferably, the composite materials are designed to have a pre-defined shape, typically a shape that is suitable for implantation into a subject as a replacement for damaged or deteriorated tissue. The particular shape and size of individual composite materials according to the invention can be selected by the practitioner to achieve any number of goals.

Composite materials according to the invention typically include pores being sufficiently large that at least oxygen can pass through. Preferably, at least some of the pores are of sufficient size that at least a portion of an animal cell can enter. At a basic level, pores disposed in the composite materials are provided to allow for oxygen to travel through the material as it is being fabricated. High levels of oxygen provided to the microorganisms producing the composite material stimulate the microorganisms to lay down fibrils in an oriented fashion, at least to some extent. The resulting oriented cellulosic material is similar in appearance and function to naturally produced cartilagenous tissue in animals.

The cellulosic component and the hydrogel component are held together through molecular interactions, such as Van der Waals forces, hydrogen bonding, or covalent cross-linking.

The nanocomposite materials of the invention are produced in accordance with the methods of the invention. The methods include the use of a microorganism, such as Acetobacter xylinum, to extracellularly lay down a cellulosic layer of material under certain growth conditions, and to extracellularly lay down a hydrogel layer of material under different growth conditions. The key growth condition that has been found to be important for this differential production of material is the oxygen level. Specifically, bacteria grown under relatively high oxygen tensions (more than 21%) substantially, almost exclusively, or exclusively produce cellulosic material comprised of β-1→4-glucan, whereas bacteria grown under relatively low oxygen tensions (20% or lower) substantially, almost exclusively, or exclusively produce hydrogel material comprised of copolymers of β-1→2-glucan, β-1→3-glucan, and/or β-1→4-glucan. As such, the cellulosic material can include β-1→4-glucan units as the majority of the units present, such as 51%, 75%, 90%, 95% 99% or greater. Accordingly, the cellulosic layer can consist of β-1→4-glucan units (i.e., 100%), consist essentially of β-1→4-glucan units (i.e., include such units as the only substance providing structure and function according to the invention), or comprise a useful portion of the layer. Likewise, the hydrogel material can include β-1→2-glucan, β-1→3-glucan, and/or β-1→4-glucan copolymers as the majority of the units present, such as 51%, 75%, 90%, 95% 99% or greater. Accordingly, the hydrogel layer can consist of β-1→2-glucan, β-1→3-glucan, and/or β-1→4-glucan copolymer units (i.e., 100%), consist essentially of such copolymer units (i.e., include such units as the only substance providing structure and function according to the invention), or comprise a useful portion of the layer. It is to be noted that all ranges discussed herein, and all ranges defined by two or more specifically recited points, include each and every value within the ranges, without the need for each value to be recited specifically herein.

The method of the invention uses control of the oxygen available to the microorganism to control the type of material produced and included in the composite material. The method of the invention also uses control of the oxygen available to the microorganism to control, at least to some extent, the orientation of cellulosic fibrils produced and incorporated into the composite material. While the order in which the two different components are produced and incorporated into the composite material is not critical, where modeling of cartilage tissue is desired, it is preferable that the method include a first step of providing relatively high levels of oxygen to the bacteria, followed by a subsequent step of providing relatively low levels of oxygen to the bacteria. The times for each of these steps will vary depending on the culture conditions and size of each layer that is desired. As a general guide, the following parameters can be used: The typical fermentation condition includes suitable nutrient medium having a pH between 3.5 and 5.5 and temperature between 20° C. and 30° C. A suitable nutrient medium is for example Schram&Hestrin medium comprising about 20 g/l glucose, 5 g/l peptone, 5 g/l yeast extract, 5 g/l yeast extract, 2,7 g/l anhydrous dibasic sodium phosphate and 1.15 g/l citric acid monohydrate. Typical fermentation time is between 3 days and 7 days.

In embodiments, the method of producing a composite material according to the invention includes: providing a support for growth of a microorganism and for delivery of oxygen to the microorganism; exposing a collection of microorganisms to the support under conditions that allow for attachment of the microorganisms to the support; providing conditions suitable for growth of the microorganisms on the support; providing a first level of oxygen to the microorganisms to cause the microorganisms to produce a particular extracellular substance; and providing a second level of oxygen to the microorganisms to cause the microorganisms to produce a different particular extracellular substance. In embodiments, the first particular extracellular substance is at least one β-1→4-glucan unit, and the second particular extracellular substance is at least one copolymer of β-1→2-glucan, β-1→β-glucan, and/or β-1→4-glucan. The method may further include removing the microorganism from the composite material after both layers have been deposited. The method may further include cross-linking or otherwise modifying one or both layers of the composite material. The method may also include washing, cleaning, and/or sterilizing the composite material after production.

In various embodiments, the method is a method of making a β-glucan nanocomposite, wherein the method includes making perpendicular cellulose nanofibers around porous glass capillaries that deliver oxygen and making a hydrogel, in which the cellulose nanofibers and hydrogel are made by one or more bacteria. The method includes controlling the oxygen supply or tension to the bacteria to produce the cellulose nanofibers and hydrogel. According to embodiments, the oxygen is delivered in pre-determined varying amounts along the length and/or width of the nanofibers. In certain embodiments, the invention provides a method of making a β-glucan nanocomposite, wherein the method includes making randomly distributed cellulose nanofibrils by exposing a bacterium to varying levels of oxygen deficiency to yield a layer of randomly distributed cellulose nanofibrils, followed by or preceded by exposing the bacterium to a lower level of oxygen to yield a layer of hydrogel, which can be a β-glucan hydrogel. The hydrogel, when crosslinked, can exhibit compression resistance.

Yet again, the invention provides a method of making a β-glucan nanocomposite where targeted oxygen delivery provides a high concentration of cellulose nanofibrils, wherein the oxygen delivery is at an oxygen tension above 21%. The invention likewise provides a method of making a β-glucan nanocomposite comprising microporous edges, wherein the edges are modified with anionic polysaccharides that induce crystallization of hydroxyapatite. The invention further provides embodiments that include a method of making a β-glucan nanocomposite in which at least the hydroxyapatite contained in the nanocomposite stimulates migration of cells and bone formation. As such, the method can include seeding one or more bacterial cells onto a solid support; exposing the cells to conditions supporting growth; and modulating or adjusting the oxygen availability to some or all of the cells to adjust growth and production of at least β-glucan, wherein the adjusting or modulating is performed to improve production of hydroxyapatite to allow for migration of osteogenic or other bone cells into the nanocomposite and production of bone formation. In another exemplary embodiment, the method provides a method of making a β-glucan nanocomposite, wherein the method comprises seeding at least one bacterium on a solid support, providing conditions under which the bacterium can grow and produce β-glucan, and adjusting oxygen levels along the solid support to provide conditions that result in different physical characteristics for the β-glucan, which results in a nanocomposite material having different properties in different layers.

The composite materials of the invention can be used in methods of treating subjects in need of replacement therapy for meniscus, cartilage, or bone injury or degeneration. Broadly speaking, the methods comprise implanting a composite material according to the invention into the subject at the site of injury or degeneration. Implanting can be accomplished by any suitable technique, and those of skill in the art of surgery are free to select any appropriate technique. While not encompassed specifically by the steps of this method of the invention, the method of treating results in replacement or augmentation of tissue of the subject. Under some circumstances, implanting of a composite material into a subject can result in invasion of certain cells of the subject into the implanted composite material, such as, for example, colonization of the implant by bone cells.

In embodiments, the method of treating is a method of treating animals and/or humans who suffer from a meniscus defect by implantation of β-glucan nanocomposite meniscus implant. The implant is a composite material according to the invention, comprising both a cellulosic component and a hydrogel component. In other embodiments, the method is a method of treating animals and/or humans who suffer from cartilage damage by implantation of β-glucan nanocomposite cartilage implant according to the invention.

### EXAMPLES

The invention will be further explained by the following Examples, which are intended to be purely exemplary of the invention, and should not be considered as limiting the invention in any way.

### Example 1

### Biomimetic Design of Cartilage

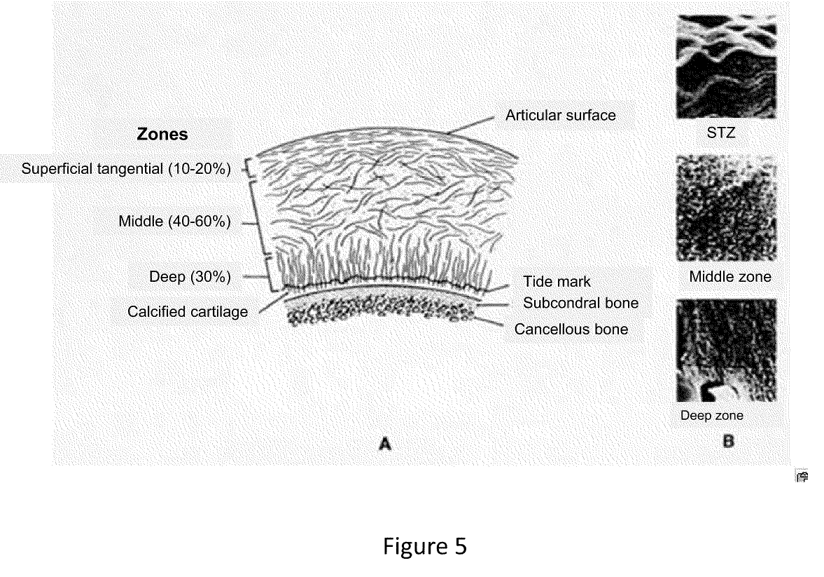

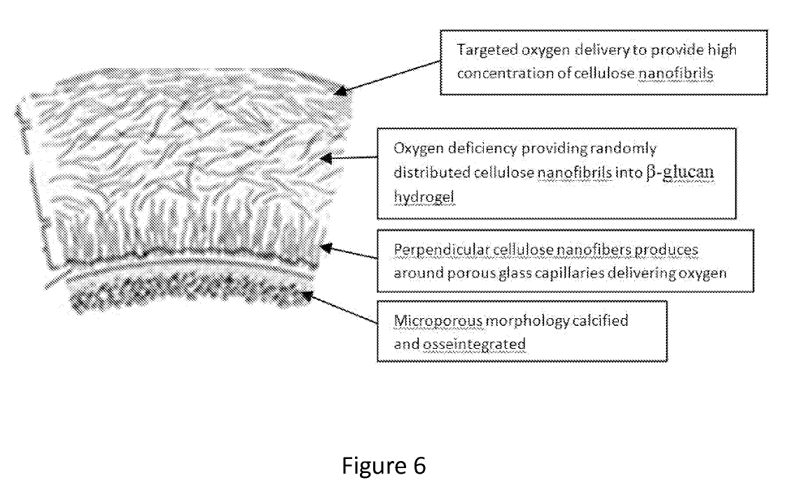

Cartilage tissue is composed of collagen nanofibrils, proteoglycans, glycoproteins, and chondrocytes. The major component is collagen. Cartilage has high water content (about 70%). FIG. 5 shows the morphology of cartilage with 4 different zones visible. FIG. 6 shows how control of bacterial fermentation can be used for preparation of cartilage replacements. Four different zones similar to native meniscus design can be prepared by designing a bioreactor that will affect the fermentation process. Using a bioreactor that has a solid substrate that is porous to oxygen, one can supply a regulated amount of oxygen to cells growing on the bioreactor, which allows high concentrations of cellulose to be produced. For example, high concentrations of cellulose can be produced close to porous glass capillaries that deliver oxygen to Acetobacter xylinum bacteria growing on the substrate that the capillaries form a part of. The high concentration of cellulose provides good mechanical properties. The oxygen deficiency inside the material produces non-cellulosic polymers. These can be cross-linked after the fermentation process. Such bacterially-produced polymers provide good compression resistance. Briefly, the different layers are produced as follows: the bacteria consume oxygen to produce cellulose. When they are left in static culture without addition of extra oxygen the oxygen deficiency will appear and the production switches.

Fermentation by Acetobacter xylinum to produce a BC meniscus was carried out using a bioreactor composed of a porous silica support with attached perpendicular porous glass capillaries. The perpendicular capillaries were provided to allow oriented growth of bacteria, and thus oriented deposition of cellulosic material, as a result of oxygen availability through the capillaries. An oxygen concentration above 21% was delivered through the oxygen-permeable material. A complex media (CSL) was used as the growth media. Glucose and fructose consumption and pH were measured using a standard enzymatic kit. The strain used for the biosynthesis was Acetobacter xylinum subsp. sucrofermentas BPR2001, tradenumber: 1700178™ from the American Type Culture Collection.

Six cellulose-forming colonies were cultivated for 2 days in Rough flasks targeting a cell concentration of 3.7×10⁶ cfu/ml in a total volume of 500 ml. The bacteria were liberated from the resulting BC hydrogel by vigorous shaking and 2.5 ml of the bacterial suspension was added to each fermentation bioreactor. The fermentation was completed after 7 days and 13-glucan nanocomposite was cross-linked using glutaraldehyde and then purified by treating in 0.1 M NaOH, 60° C., for 4 hours and thereafter repeated boiling in Millipore™ water. The β-glucan nanocomposite was steam sterilized by autoclaving for 20 minutes at 120° C., 1bar, and stored in a refrigerator until further use.

More specifically, in the rough flask the BC hydrogel pre-culture is grown. That is a way to proliferate bacteria. Then a portion of the bacteria suspension is added to a bioreactor in the form of for example meniscus. This bioreactor is fitted with the porous glass capillaries to provide higher than air (more than 21% oxygen). Bacteria produce glucans layer by layer (about 1-2 mm per day). These layers are composed of cellulose nanofibrils (if there is no higher oxygen tension). When oxygen tension is increased more cellulose is produced and then less oxygen is available which result in oxygen deficiency and production of glucan hydrogel starts. This process repeats layer by layer until the bioreactor object is filled with the composite.

As a consequence of the bioreactor design for targeted oxygen delivery, β-1→4-glucan nanocomposite described in this invention has high strength, good compression resistance, perpendicular fibers, and microporosity at the edges, which has been found to be useful in stimulating the osseointegration process.

### Example 2

### Introduction of Calcium Into Scaffolds

In this study, a cartilage β-glucan nanocomposite implant was modified by adsorption of carboxymethyl cellulose, CMC, in the presence of 0.01 M CaCl₂. After CMC treatment, CMC modified (β-glucan nanocomposite at different conditions such as concentration of CMC, time of treatment, temperature of treatment was then exposed to Simulated Body Fluid (SBF) in static conditions for 1 week. The amount of crystals grown and composition was determined using XPS. It was found that pretreatment of a β-glucan nanocomposite had a large effect on crystal growth. At certain conditions, such as 1 week exposure of nanocomposite to SBF solution at 37° C., growth of nanocrystals with ratio Ca:P of 1.75 was achieved. This ratio is close to the chemical composition of hydroxyapatite (1.66).

The effect of calcium phosphate on cells was investigated with regard to osteoblastic differentiation of bone marrow stromal cells (BMSCs). It was found that the presence of hydroxyapatite (HAP) enhanced deposition of the osteopontin (OPN) protein. The data show that osteoblastic cells are able to adhere to and proliferate on HAP and cellulose surfaces. Overall, these data demonstrate that hydroxyapatite surfaces are better for adhesion and proliferation of osteoblastic cells.

### Example 3

### Mimicking Meniscus Morphology Using Bacteria to Produce

Implants

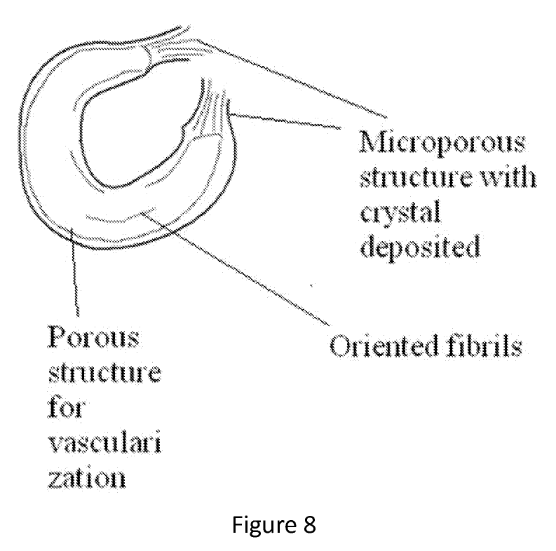

Meniscus has the major function of providing stability for the knee joint and protecting cartilage from degeneration (e.g., osteoarthritis). Meniscus is composed mostly of collagen fibrils and water. There are two types of collage fibrils, collagen I and II. The major contribution to the mechanical properties of the meniscus is due to the presence of circumferential collagen I fibrils. The meniscus is attached to the bones through the horns of the meniscus. A bioreactor was designed (see FIG. 8) in which a meniscus implant based on (β-1→4-glucan nanocomposite is produced. The outer part of the meniscus has cavities that are produced as a result of growth of bacteria and deposition of glucan units around, but not within, rod-like structures present on the solid substrate. Upon removal of the structures, cavities remain, which can be used for various purposed, including as points for suturing of the composite to body tissue. The cavities can also serve to enable vascularization and promote integration of the implant into the body. Inside the bioreactor, bundles of parallel permeable oxygen delivering capillaries are placed circumferentially. These enable production of parallel cellulose fibrils. Many fibril bundles are added at the horns of the implant to produce a highly porous structure. Such a structure can be modified (see Example 2) to enable osseointegration.

As a consequence of the bioreactor design for targeted oxygen delivery, a β-1→4-glucan nanocomposite described in this disclosure has cavities at the edges, which enable vascularization and integration. It also has circumferential fibrils that give strength and microporosity at the horns, a property that can be used to stimulate osseointegration process.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

### REFERENCES

Gelin, K., Bodin, A., Gatenholm, P., Mihranyan, A., Edwards, K and Stromme, M., Characterisation of Water in Bacterial Cellulose Using Dielectric Spectroscopy and Electron Microscopy, Polymer, 48, 7623-7631 (2007)
Bodin, A., Concaro, S., Brittberg, M., and Gatenholm, P., Bacterial Cellulose as a Potential Meniscus Implant, Journal of Tissue Engineering and Regenerative Medicine, 48, 7623-7631 (2007)
Bodin, A., Backdahl, H., Fink, H., Gustafsson, L., Risberg, B., and Gatenholm, P., Influence of cultivation conditions on the mechanical and morphological properties of bacterial cellulose tubes, Biotechnology and Bioengineering, 97 (2), 425-434 (2007)
Helenius, G., Backdahl, H., Bodin, A., Nanmark, U., Gatenholm, P., and Risberg, B., In vivo Biocompatibility of Bacterial Cellulose, J. Biomed. Mater. Res. A., 76(2), 431-438 (2006) Backdahl, H., Helenius, G., Bodin, A., Johansson, B., Nanmark, U., Risberg, B., and Gatenholm, P., Bacterial Cellulose as Potential Scaffold for Tissue Engineered Blood Vessels: Mechanical Properties and Cell Interactions, Biomaterials, 27, 2141-2149 (2006)
Kobayashi M., Chang Yong-Shun., and Oka M., A two year in vivo study of PVA-H artificial meniscus, Biomaterials 26, 3243, (2005)

1. An engineered composite material comprising:
a cellulosic layer comprising β-1→4-glucan units; and
a hydrogel layer comprising copolymers of β-1→2-glucan, β-1→β-glucan, β-1→4-glucan units, or mixtures of two or all three of these units.

2. The composite material of claim 1, which is produced by bacteria.

3. The composite material of claim 1, wherein the cellulosic layer consists essentially of β-1→4-glucan units and the hydrogel layer consists essentially of copolymers of β-1-2-glucan, β-1→β-glucan, β-1→4-glucan units, or mixtures of two or all three of these units.

4. The composite material of claim 1, which shows a microporosity at its edges.

5. A method of producing a composite material, said method comprising:
culturing a microorganism on a solid substrate capable of delivering controlled amounts of oxygen to the microorganism;
providing to the microorganism a first level of oxygen to cause the microorganism to produce a first type, or combination of types, of glucan units;
providing to the microorganism a second level of oxygen to cause the microorganism to produce a second type, or combination of types, or glucan units,
wherein only a single culture of microorganisms is used and wherein the culture media for the microorganism is not altered between production of the first type(s) and second type(s) of glucan units.

6. The method of claim 5, wherein the first level of oxygen is higher than the second level of oxygen.

7. The method of claim 5, wherein the first type of glucan unit is a β-1→4-glucan unit.

8. The method of claim 5, wherein the second type of glucan unit is a mixture of copolymers of β-1→2-glucan, β-1→β-glucan, or β-1→4-glucan units, or mixtures of two or all three of these units.

9. The method of claim 8, further comprising cross-linking the copolymer units.

10. The method of claim 5, wherein production of the first type of glucan unit results in deposition of cellulosic fibrils on the solid substrate.

11. The method of claim 5, wherein production of the second type of glucan unit results in production of a hydrogel.

12. The method of claim 5, which is a method of producing a composite material comprising a strong, rigid cellulosic layer and a compressible hydrogel layer.

13. The method of claim 5, which is a method of producing engineered meniscus or engineered cartilage tissue.

14. A method of treating a subject suffering from injury or degeneration of meniscus or cartilage, said method comprising:
implanting an engineered meniscus or cartilage replacement composite material into the subject at the site of injury or degeneration,
wherein the composite material comprises a first cellulosic layer and a second hydrogel layer.

15. The method of claim 14, wherein the composite material includes structures for attachment of the material to tissues of the subject.

16. The method of claim 14, wherein the composite material includes structures for infiltration of cells from the subject into the material.
